(19) United States
(12) Patent Application Publication
(10) Pub. No.: US 2025/0009407 A1
(43) Pub. Date: Jan. 9, 2025

(54) FLUID CONNECTION APPARATUS AND METHOD OF USE

(71) Applicant: INTUITIVE SURGICAL OPERATIONS, INC., Sunnyvale, CA (US)

(72) Inventors: Cassandra R. Saleira, Bothell, WA (US); Jacqueline L. Corso, Kirkland, WA (US); Erik M. Henne, Seattle, WA (US); Alexander M. Vanden Bussche, Seattle, WA (US)

(21) Appl. No.: 18/761,088

(22) Filed: Jul. 1, 2024

Related U.S. Application Data
(60) Provisional application No. 63/511,839, filed on Jul. 3, 2023.

(51) Int. Cl. A61B 18/04; A61B 18/00; F16L 39/00

(52) U.S. Cl. CPC A61B 18/04; F16L 39/00; A61B 2018/00095; A61B 2018/00101; A61B 2018/00172; A61B 2018/00577; A61B 2018/00791; A61B 2018/046

(57) ABSTRACT

A connector assembly may comprise a connector housing and a connector manifold disposed within the connector housing. The connector manifold may include a manifold body, a first channel extending through the manifold body to a first chamber, a second channel extending through the manifold body to a second chamber, and a third channel extending through the manifold body to a third chamber, wherein the first chamber, the second chamber, and the third chamber are coaxial.

### CROSS-REFERENCED APPLICATIONS

This application claims priority to and benefit of U.S. Provisional Application No. 63/511,839 filed Jul. 3, 2023 and entitled “Fluid Connection Apparatus and Methods of Use,” which is incorporated by reference herein in its entirety.

### FIELD

Examples described herein relate to a fluid connection apparatus that provides an interface between a thermal fluid generation system and an instrument such as a catheter.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. Some minimally invasive medical instruments may be used to perform ablation or other thermal treatments. In some examples, minimally invasive instruments may convey a heated fluid from a thermal fluid generation system to a treatment location to perform a thermal treatment. Improved systems and methods are needed to efficiently and effectively couple instruments to the thermal fluid generation system.

### SUMMARY

The following presents a simplified summary of various examples described herein and is not intended to identify key or critical elements or to delineate the scope of the claims.

In some examples, connector assembly may comprise a connector housing and a connector manifold disposed within the connector housing. The connector manifold may include a manifold body, a first channel extending through the manifold body to a first chamber, a second channel extending through the manifold body to a second chamber, and a third channel extending through the manifold body to a third chamber, wherein the first chamber, the second chamber, and the third chamber are coaxial.

In some examples, a catheter assembly may comprise a catheter body and a catheter hub assembly coupled to a proximal end of the catheter body. The catheter hub assembly may include a manifold body including a central fluid channel, a first flange concentric with the central fluid channel, a second flange proximal to the first flange and concentric with the central fluid channel, and a third flange proximal to the second flange and concentric with the central fluid channel. The catheter hub assembly may also include a first channel extending through the manifold body between the first flange and the central fluid channel and a second channel extending through the manifold body between the second flange and the central fluid channel.

In some examples, a fluid connection apparatus may comprise a connector assembly configured to connect with a thermal fluid generation system. The connector assembly may include a first fluid channel and a second fluid channel. The fluid connection apparatus may also comprise a hub assembly configured to connect with an ablation tool. The hub assembly may comprise a third fluid channel connected to the first fluid channel and a fourth fluid channel connected to the second fluid channel. The hub assembly and the connector assembly may be configured to rotate with respect to each other while maintaining connections for fluid transfer between the first and the third fluid channels and between the second and forth fluid channels.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates a medical instrument system extended within a patient anatomy, according to some examples.

FIG. 2A illustrates a fluid connection apparatus, according to some examples.

FIG. 2B illustrates an exploded view of the fluid connection apparatus of FIG. 2A.

FIGS. 3A and 3B illustrate perspective views of a connector assembly, according to some examples.

FIG. 3C illustrates a perspective view of a connector manifold, according to some examples.

FIG. 3D illustrates a cross-sectional view of the connector manifold of FIG. 3C.

FIG. 3E illustrates a distal end view of the connector manifold of FIG. 3C.

FIG. 4A illustrates a side view of a catheter hub assembly, according to some examples.

FIG. 4B illustrates a cross-sectional view of the catheter hub assembly of FIG. 4A.

FIG. 4C illustrates a perspective view of a hub manifold, according to some examples.

FIG. 4D illustrates a side view of the hub manifold of FIG. 4C.

FIG. 4E illustrates a cross-sectional view of the hub manifold of FIG. 4C.

FIG. 5A illustrates a cross-sectional view of the fluid connection apparatus of FIG. 2A.

FIG. 5B illustrates a cross-sectional view of the fluid connection apparatus of FIG. 2A.

FIG. 6 illustrates a cross-sectional view of a catheter hub assembly, according to some examples.

FIG. 7 illustrates a cross-sectional view of a connector assembly, according to some examples.

FIG. 8 is a robot-assisted medical system, according to some examples.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

The technology described herein provides techniques and treatment systems for endoluminal thermal treatment of diseased tissue. The thermal treatment may be provided by fluid delivered from a thermal fluid generation system to the diseased tissue through an elongate instrument. Various examples are provided of a fluid connection apparatus that may provide an interface between a thermal fluid generation system and the elongate instrument. The fluid connection apparatus may connect multiple fluid receipt and/or return channels in the instrument to thermal fluid generation system with a single coupler.

A single coupler may eliminate or reduce inefficiencies associated with systems that require separate couplings for each fluid channel. For example, some systems may include separate tubes from the thermal fluid generation system to provide a heated treatment fluid to the elongate instrument, to provide a heated circulation fluid to the elongate instrument, and to remove fluid from the elongate instrument. The length of the tubes may result in a longer, less efficient fluid flow pathway and may allow more surface area for heat loss. Furthermore, the separate tubes may require tedious and time consuming connection and disconnection to the elongate instrument to introduce the heated fluids and/or remove the cooled fluid. Multiple fluid tubes may also introduce the opportunity for error in mis-connecting a tube.

The systems and methods described herein provide a connector assembly that couples to the thermal fluid generation system. A hub assembly is coupled to the elongate instrument. The hub assembly is connectable to the connector assembly with features that may reduce or minimize the inefficiencies and/or risks associated with existing systems. For example, heat loss may be minimized by using connector and hub manifolds that may be heated by the heated fluid to help maintain the temperature of the flowing fluids. The heated fluid channels may be separated from the cooled fluid channels in the manifolds to insulate the heated fluid from the cooled, returning fluid and reduce any parasitic heat-loss caused by the cooler fluid. The length of the channels through the manifolds may also be minimized, compared to separate tubes, to reduce the distance along which heat loss may occur. The connector assemblies described herein may include coaxial fluid chambers which may reduce or eliminate the need for directionally keyed connecting pieces, allowing rotation or introduction in any rotational direction (e.g., in any of 360 degrees of orientation). The coaxial fluid chambers, separated by seals, may also allow the catheter hub assembly to rotate relative to the connector assembly while the connector assembly remains fixedly connected to the thermal fluid generation system and separation is maintained between the different fluids. Various sensors and interlock features may create a secure connection between the connector assembly and the hub assembly, minimizing the risk of separation caused by high pressure fluids and minimizing the risk to users terminating fluid flow if disconnection occurs.

Although the examples provided herein may refer to treatment of lung tissue and pulmonary disease, it is understood that the described technology may be used in treating artificially created lumens or any endoluminal passageway or cavity, including in a patient trachea, colon, intestines, stomach, liver, kidneys and kidney calices, brain, heart, circulatory system including vasculature, fistulas, and/or the like. In some examples, treatment described herein may be referred to as endobronchial thermal fluid treatment and may be used in procedures to treat lung tumors and/or chronic obstructive pulmonary disease (COPD) that may include one or more of a plurality of disease conditions including chronic bronchitis, emphysema, and bronchiectasis.

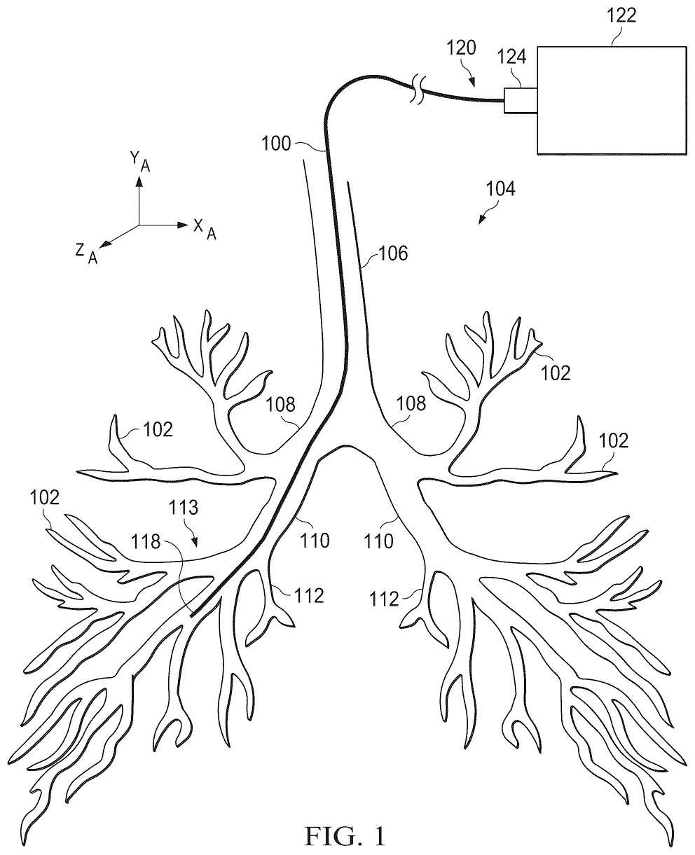

FIG. 1 illustrates an elongated medical instrument system 100 extending within branched anatomic passageways or airways 102 of an anatomical structure 104. In some examples the anatomic structure 104 may be a lung and the passageways 102 may include the trachea 106, primary bronchi 108, secondary bronchi 110, and tertiary bronchi 112. The anatomic structure 104 has an anatomical frame of reference (X_{A}, Y_{A}, Z_{A}). A distal end portion 118 of the medical instrument system 100 may be advanced into an anatomic opening (e.g., a patient mouth) and through the anatomic passageways 102 to perform a medical procedure, such as an endoluminal thermal energy treatment, at or near target tissue located in a region 113 of the anatomic structure 104. A proximal portion 120 of the medical instrument system 100 may be coupled to a thermal fluid generation system 122 by a fluid connection apparatus 124. The elongated medical instrument system 100 may be used for ablating diseased tissue. For example, a heated fluid may flow from the thermal fluid generation system 122, through the fluid connection apparatus 124, and through the elongated medical instrument system 100 to perform a thermal treatment, such as an ablation, of tissue in the region 113. In some examples, the thermal fluid generation system 122 may heat fluid with resistive heating, radiofrequency heating, ultrasonic heating, laser heating, magnetic heating, and/or microwave heating.

During ablation, cellular and structural changes in the epithelium and sub-epithelium may be induced. The ablation may cause tissue reduction, including destruction of goblet cells and cilia in lung tissue. In some examples, the cellular matrix may be preserved to allow for later regrowth of healthy cells. In some examples, the tissue reaction may occur entirely during the application of the heat, and in other examples, the tissue damage may develop over a period of time as the anatomy responds to the injury caused by the heat. In the lung, an ablation may be used to treat a variety of pulmonary conditions including lung tumors, chronic obstructive pulmonary disease, chronic bronchitis, emphysema, and bronchiectasis.

An endoluminal thermal energy treatment system, such as an endoluminal ablation system, may deliver a heated treatment fluid through a catheter system directly to the patient anatomy or to an ablation device, such as a balloon that expands to engage a tissue surface in the patient anatomy. In some examples, a heated circulating fluid may circulate (e.g. flow into and out of) through the catheter system to, for example, pre-heat the ablation device before and/or while the heated treatment fluid is delivered or maintain the temperature of the heated treatment fluid as it travels within the catheter and the ablation device. Examples of ablation systems for endoluminal thermal energy treatment that use a heated circulating fluid and a heated treatment fluid are described, for example, in U.S. Provisional Application 63/299,317 filed Jan. 13, 2022 describing “Systems and Methods for Localized Ablation with a Circulating Fluid Catheter,” which is incorporated by reference herein, in its entirety. International Publication No. WO 2022/140287 A1 filed Dec. 20, 2021 describing, “Systems for Temperature Control in Delivery of Thermal Liquid Treatment,” is also incorporated by reference herein, in its entirety. In some examples, the instruments and systems of this disclosure may be used without a circulation system during the delivery of a heated treatment fluid.

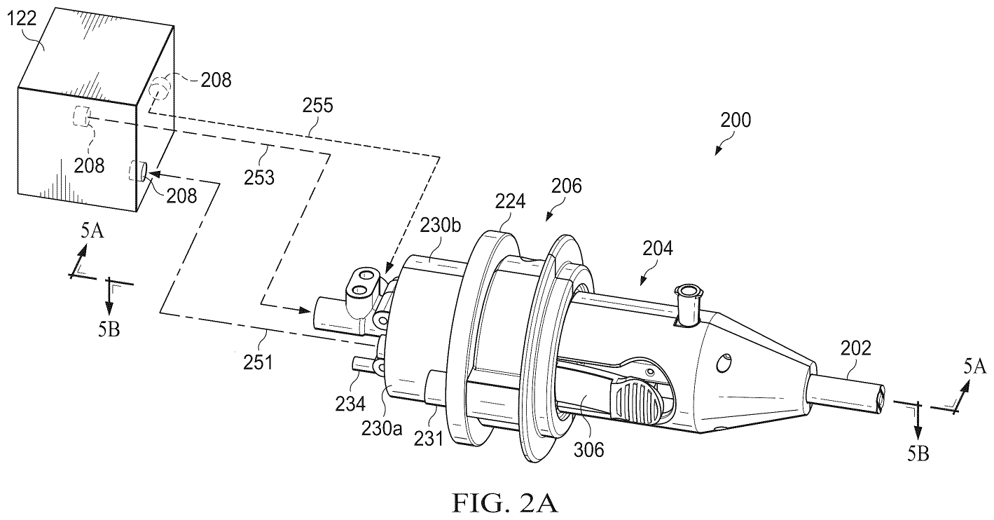

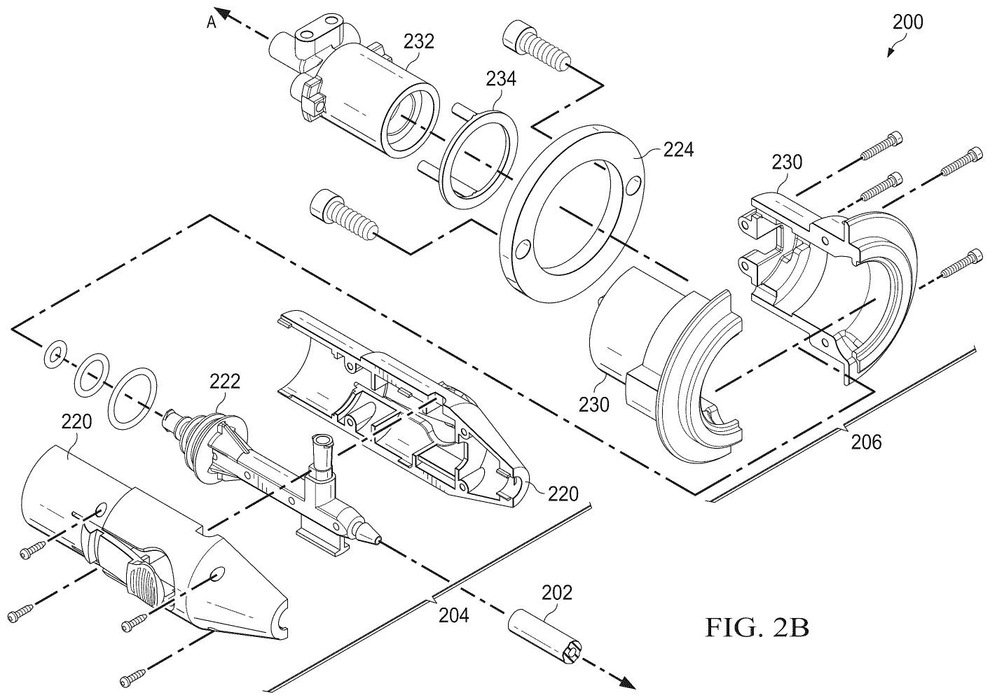

FIG. 2A illustrates a fluid connection apparatus 200 which may be used as the fluid connection apparatus 124 to provide a coupling between the thermal fluid generation system 122 and a flexible catheter 202 of the elongated medical instrument system 100, or some other type of ablation tool that uses thermal fluid. The fluid connection apparatus 200 may connect the medical instrument system 100 to the thermal fluid generation system 122 to deliver a heated treatment fluid 253 and a heated circulating fluid 255 to the catheter 202 and to pass or drain a return fluid 251 (which may include the fluid 255 and/or the fluid 253) from the catheter 202 to the thermal fluid generation system 122. The fluids 251, 253, 255 may include any of a variety of different fluid substances including water (e.g., distilled water), saline, or a gas such as air. In some examples, the fluid 201 may include a radiopaque substance. The substances listed above are not limiting, but examples.

The fluid connection apparatus 200 may include a hub assembly 204 that connects to a connector assembly 206. The hub assembly 204 may be coupled to the proximal end portion of the flexible catheter 202. The connector assembly 206 may be coupled to a plurality of ports 208 of the thermal fluid generation system 122 to convey fluid to and/or from the thermal fluid generation system 122.

FIG. 2B is a view of the fluid connection apparatus 200 exploded along a longitudinal axis A. The hub assembly 204 may include a hub housing 220 and a hub manifold 222. In some examples, the hub housing 220 may include two connectable halves as shown in FIG. 2B. The connector assembly 206 may include a connector housing 230, a connector manifold 232, and an interlock assembly 234. In some examples, the connector housing 230 may include two connectable halves as shown in FIG. 2B.

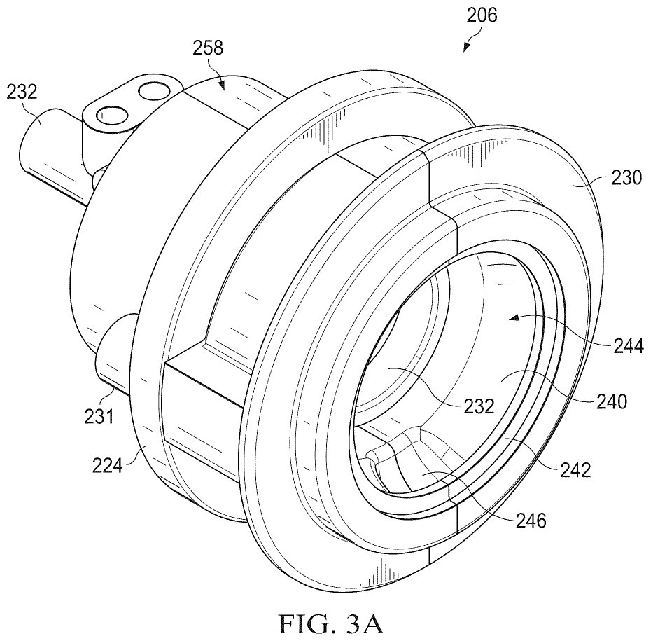

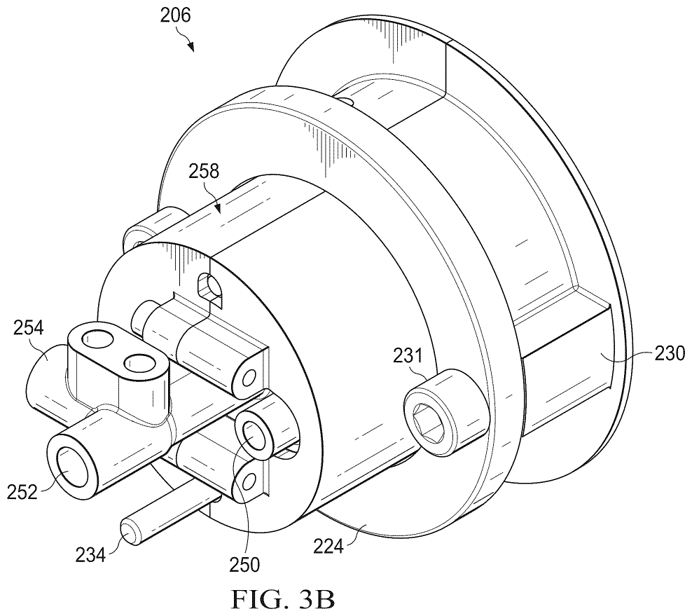

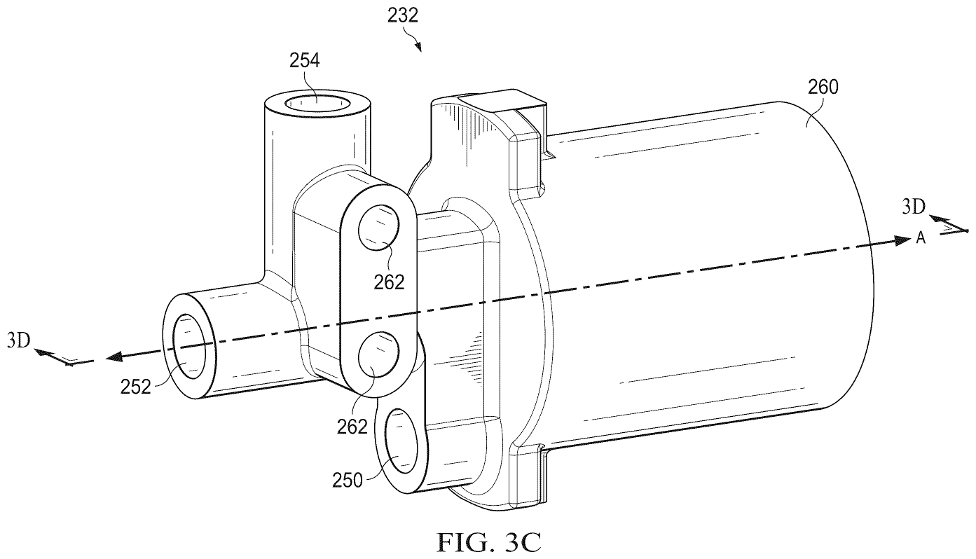

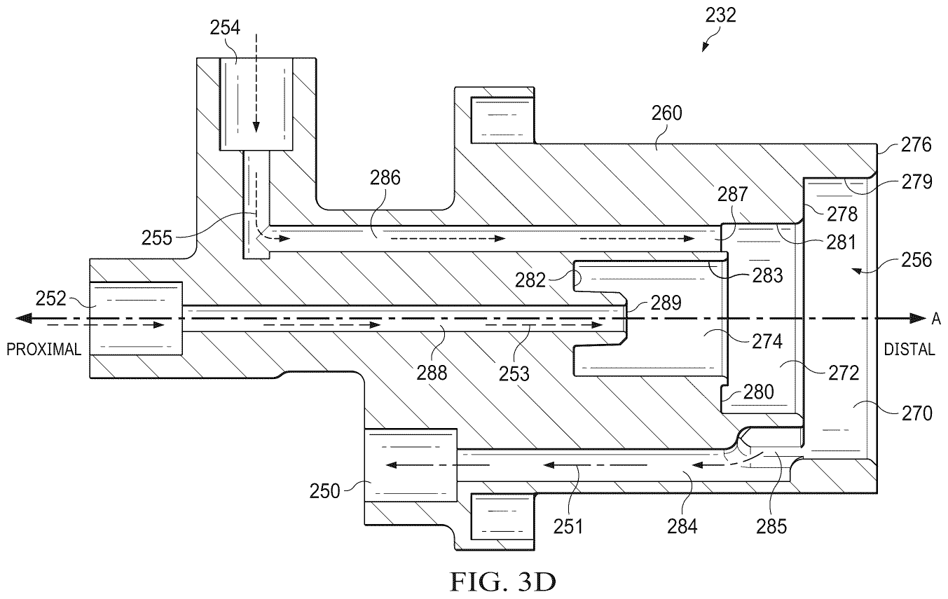

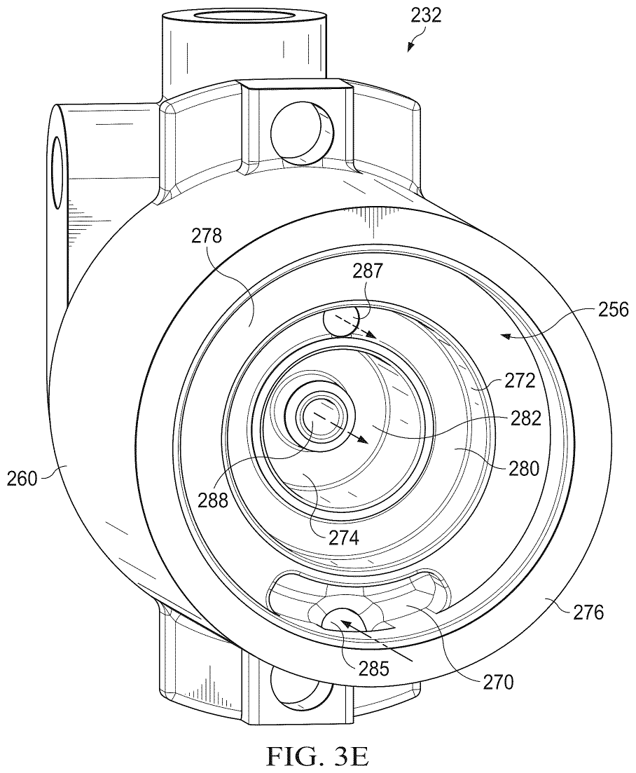

FIGS. 3A and 3B are perspective views of the assembled connector assembly 206. The connector housing 230 may include a central passage 240 with a flange 242 extending around a distal opening 244 to the central passage 240. A drip port 246 may extend through the connector housing 230 near the flange 242 to allow fluid to escape from the fluid connection apparatus 200 during the connection or disconnection of the connector assembly 206 and the hub assembly 204. The connector housing 230 may at least partially encase the connector manifold 232 such that a central chamber 256 (see FIG. 3D) of the connector manifold 232 is generally coaxial with the central passage 240 of the connector housing. Portions of the connector manifold 232 such as fluid ports 250, 252, 254 may extend through a proximal portion 258 of the connector housing 230. In this example, the connector housing 230 may including two halves 230a, 230b that connect for case of assembly. A connector ring 224 may surround the two halves 230a, 230b of the connector housing 230 and may be secured to the connector housing 230 by attachment devices 231 such as screws. In other examples the connector housing and connector manifold may be a single integral unit or may be formed of a plurality of connectable components.

FIG. 3C illustrates a perspective view of the connector manifold 232. FIG. 3D illustrates a cross-sectional view of the connector manifold 232, and FIG. 3E illustrates a distal end view of the connector manifold 232. The connector manifold 232 may include a manifold body 260 in which the central chamber 256 extends. The fluid ports 250, 252, 254 and sensor ports 262 extend proximally from the manifold body 260. In this example the fluid ports 250, 252 may extend generally parallel to the longitudinal axis A, fluid port 254 may extend generally perpendicular to the longitudinal axis A and sensor ports 262 may extend generally perpendicular to the longitudinal axis A and generally perpendicular to the fluid port 254.

As shown in FIGS. 3D and 3E, the central chamber 256, is comprised of coaxial chambers 270, 272, 274 arranged in a stacked or tiered formation along the axis A. Chamber 270 (e.g., a first fluid chamber) extends generally from an outer surface 276 to a circumferential surface 278 and is bounded by a circumferential wall 279. Chamber 272 (e.g., a second fluid chamber) extends generally from the circumferential surface 278 to a circumferential surface 280 and is bounded by a circumferential wall 281. Chamber 274 (e.g., a third fluid chamber) extends generally from the circumferential surface 278 to a proximal wall 282 of the central chamber 256 and is bounded by a circumferential wall 283. The diameters of the chambers 270, 272, 274 may be progressively smaller in a distal to proximal direction along the axis A. That is, the fluid chamber 274 may have a smaller diameter than the fluid chamber 272, and the fluid chamber 272 may have a smaller diameter than the fluid chamber 270. A channel 284 may extend from a port 285 at the chamber 270 to the port 250 to provide a passage for return fluid 250. A channel 286 may extend from a port 287 at the chamber 272 to the fluid port 254 to provide a passage for the heated circulating fluid 255. A channel 288 may extend from an opening 289 in the fluid chamber 274 to the port 252 to provide a passage for the heated treatment fluid 253.

The arrangement of the fluid chambers 270, 272, 274 with the fluid chamber 272 (containing the circulating heating fluid 255) as an intermediary chamber, shields the heated treatment fluid 253 in chamber 274 from the cooled return fluid in chamber 270. This arrangement may use the circulating heated fluid 255 to insulate the heated treatment fluid 253 to maintain the treatment fluid temperature and reduce parasitic heat loss to the cooled return fluid 251.

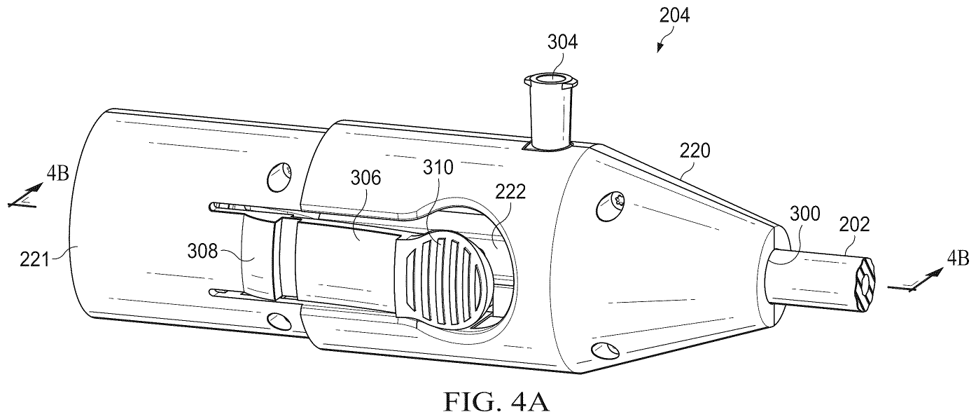

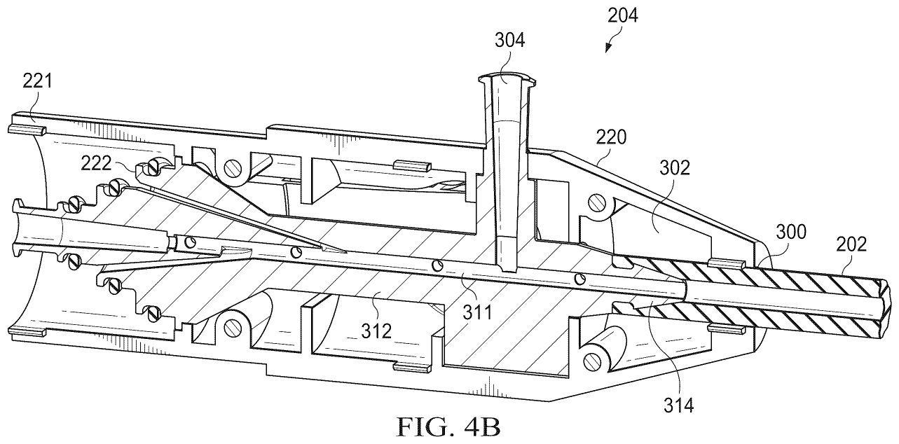

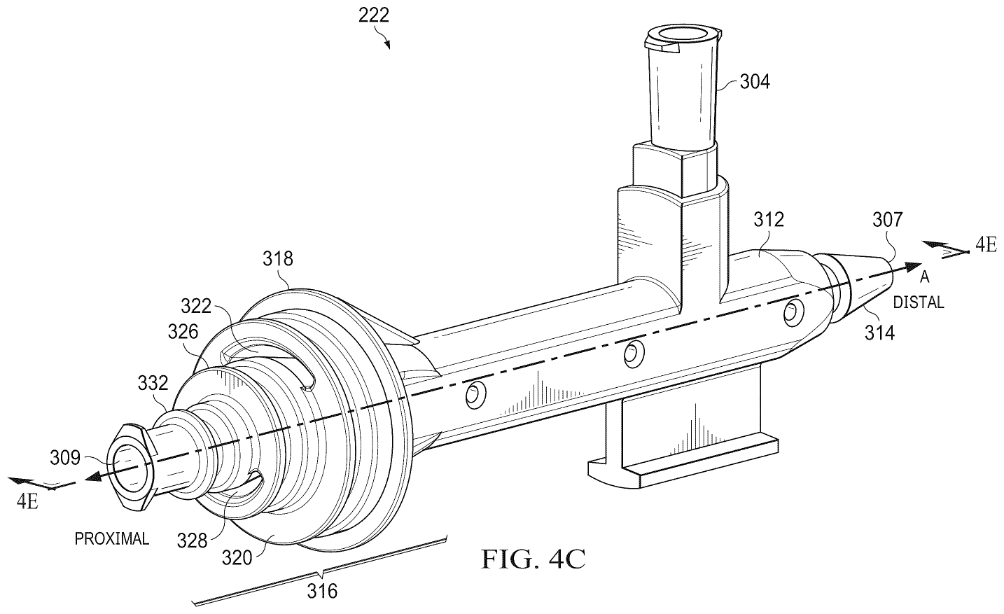

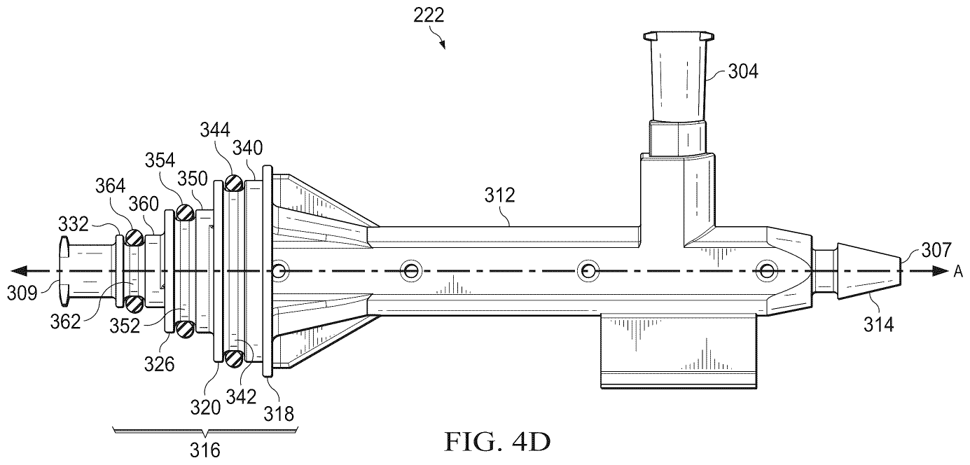

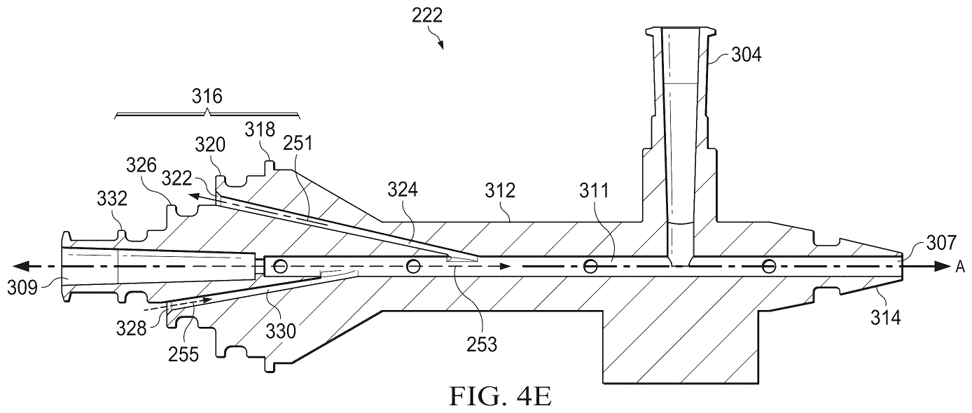

FIG. 4A is a side view of the assembled hub assembly 204, and FIG. 4B is a cross-sectional view of the assembled hub assembly 204. The hub housing 220 may include a distal opening 300 to a central region 302 in which the hub manifold 222 is positioned. An access port 304 of the hub manifold 222 may extend through a wall of the hub housing 220. The access port 304 may allow for the injection of fluid or the application of a vacuum to a central fluid channel 311 of the hub manifold. Pinch tabs 306 may extend from opposite sides of the hub housing 220 and may include engagement members 308 and gripping surface 310. In this example the hub housing may including two halves that connect (e.g., lock together) for case of assembly, but in other examples the hub housing and hub manifold may be a single integral unit or may be formed of a plurality of connectable components.

FIGS. 4C, 4D, and 4E are perspective, side, and cross-sectional views, respectively, of the hub manifold 222. The hub manifold 222 may include a hub manifold body 312 through which the central fluid channel 311 may extend. The central channel 311 may extend along or parallel to the axis A between a distal opening 307 and a proximal opening 309. A distal portion of the hub manifold body 312 may include a cannula connector member 314. The cannula connector member 314 may include a tapered nozzle or other type of connection member for press fit, frictional fit, threaded fit or other type of connection to the catheter 202. The central channel 311 may extend through the cannula connector member 314.

The hub manifold 222 may also include a sealing portion 316 which includes a series of co-axial flanges and scaling members longitudinally arranged along the axis A. Flange 318 may be a distal-most flange of the scaling portion 316 and may have the largest diameter of the flanges of the scaling portion 316. A flange 320 (e.g., a first flange) may have a smaller diameter than the flange 318 and may be spaced proximally from the flange 318 along the axis A. The flange 320 may include a fluid port 322. A channel 324 may extend through the body 312 between the fluid port 322 and the central channel 311. A flange 326 (e.g., a second flange) may have a smaller diameter than the flange 320 and may be spaced proximally from the flange 320 along the axis A. The flange 326 may include a fluid port 328. A channel 330 may extend through the body 312 between the fluid port 328 and the central channel 311. Either or both of the fluid ports 322, 328 may have an elongated arcuate shape. In other examples, the fluid ports may be circular or another suitable shape. In this example, the channels 324 and 330 may form acute angles with the central channel 311. In some examples, the fluid port 322 may be positioned approximately 180 degrees opposite the fluid port 328 about the axis A. A flange 332 (e.g., a third flange) may have a smaller diameter than the flange 326 and may be spaced proximally from the flange 326 along the axis A. The proximal opening 309 may be spaced proximally from the flange 332 along the axis A.

As shown in FIG. 4D, the sealing portion 316 may include a circumferential wall 340 extending between the flange 320 and the flange 318. A circumferential groove 342 in the circumferential wall 340 may be sized to receive a sealing member 344, such as a flexible O-ring, that provides a seal at the flange 320. The sealing portion 316 may also include a circumferential wall 350 extending between the flange 326 and the flange 320. A circumferential groove 352 in the circumferential wall 350 may be sized to receive a scaling member 354, such as a flexible O-ring, that provides a seal at the flange 326. The scaling portion 316 may also include a circumferential wall 360 extending between the flange 332 and the flange 326. A circumferential groove 362 in the circumferential wall 360 may be sized to receive a scaling member 364, such as a flexible O-ring, that provides a seal at the flange 332. Because of the graduated diameters of the flanges 318, 320, 326, 332, the sealings members 344, 354, 364 may likewise have progressively smaller diameters in a distal to proximal direction.

In use and as shown in FIG. 2A, the connector assembly 206 may be coupled to the ports 208 of the thermal fluid generation system 122 to convey fluid to and/or from the thermal fluid generation system. For example, the fluid port 252 may be coupled to the thermal fluid generation system 122 to receive the heated treatment fluid 253 into the channel 288. The fluid port 254 may be coupled to the thermal fluid generation system 122 to receive the heated circulating fluid 255 into the channel 286. The fluid port 250 may be coupled to the thermal fluid generation system 122 pass the return fluid 251 from the channel 284 to the thermal fluid generation system 122. The hub assembly 204 may be coupled to the catheter 202 by inserting the cannula connector member 314 into the catheter so that the central channel 311 of the hub manifold 222 is connected to the catheter to convey fluid into and out of the catheter.

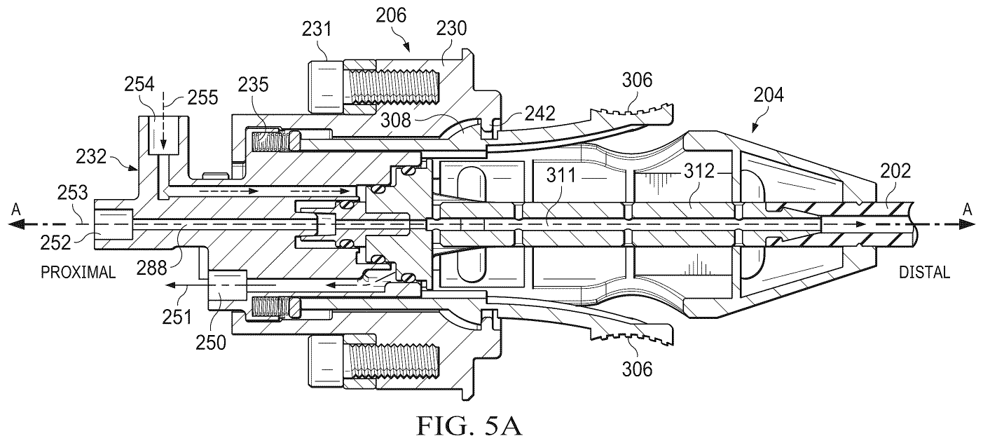

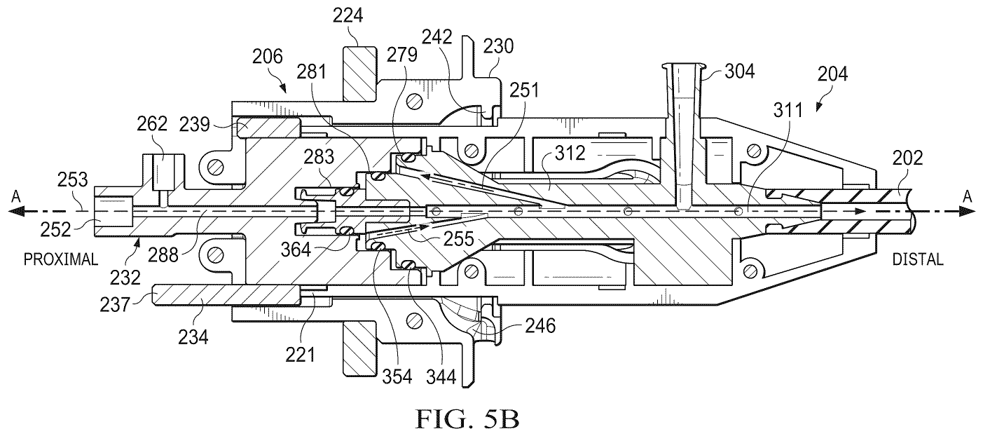

The connector assembly 206 may be coupled to the hub assembly 204 as shown in FIG. 2A and the cross-sectional views of FIGS. 5A and 5B. An operator may press the pinch tabs 306 together as the sealing portion 316 of the hub assembly 204 is inserted through the distal opening 244 and into the central passage 240 of the connector assembly 206. When the pinch tabs 306 are released, the engagement members 308 on the pinch tabs 306 may engage with the flange 242 of the connector assembly 206 to prevent separation of the hub assembly 204 relative to the connector assembly 206, while allowing the hub assembly 204 to rotate about the axis A with respect to the connector assembly 206. When connected, the scaling member 344 may engage with the circumferential wall 279, and the sealing member 354 may engage with the circumferential wall 281 to seal the boundaries of the fluid chamber 270. Further, when connected, the scaling member 364 may engage with the circumferential wall 283 such that the scaling members 354, 364 seal the boundaries of the fluid chamber 272. As connected, the fluid port 322 to the fluid channel 324 may be open to and in fluid communication with the fluid chamber 270. As connected, the fluid port 328 to the fluid channel 330 may be open to and in fluid communication with the fluid chamber 272. As connected, the fluid channel 288 of the connector assembly 206 may be aligned with and in fluid communication with the central channel 311 of the hub assembly 204. In some examples, the connector assembly 206 and the coupled hub assembly 204 may be used without fluid flowing in the fluid channels 324, 330 or the fluid chambers 270, 272. In such examples, the channels and chambers may serve as a safety mitigation to reduce the risk of delivery fluid leakage in the event of a seal failure.

While the connector assembly 206 is coupled to the hub assembly 204, the hub assembly 204 (and the coupled catheter 202) may be rotatable about the axis A relative to the connector assembly 206. The rotary connection maintains fluid separation with a rotary seal of the chambers 270, 272, 274 by the sealing members 344, 354, 364. The coaxial, sealed fluid chamber design may prevent the fluids 251, 253, 255 from migrating or leaking as the hub assembly rotates relative to the connector assembly. The rotatable coupling may also reduce stress at the connection between the catheter and the thermal fluid generation system and may prevent damage to the catheter resulting from such stress. The coaxial fluid chambers may also reduce or eliminate the need for directionally keyed or directionally mating manifolds, allowing rotation or introduction of the hub assembly and catheter in any rotational direction (e.g., in any of 360 degrees of orientation) about the axis A.

The spring-loaded interlock assembly 234 may include a long prong 237 and a short prong 239. The interlock assembly may include springs 235 (see FIG. 5A) that bias the prongs 237, 239 in a distal direction. When a proximal portion 221 of the hub housing 220 engages the interlock assembly 234, the springs 235 may be compressed and the prongs 237, 239 are moved proximally relative to the connector manifold 232. The movement of the interlock assembly may activate a sensor (e.g., an electromagnetic sensor) to indicate or confirm a secure connection between the connector assembly 206 and the hub assembly 204. In some examples the longer prong 237 may be used to guide the connection to the thermal fluid generation system and the shorter prong 239 may actively engage the sensor. The sensor may provide data to a control system (e.g., a control system 812) to prevent fluid flow if the connector assembly 206 and the hub assembly 204 become disconnected.

With the connector assembly 206 coupled to the hub assembly 204, the heated circulating fluid 255 may be introduced through the fluid port 254 in the connector assembly and may flow through the channel 286 in the connector manifold 232 into the circumferentially scaled fluid chamber 272. The heated circulating fluid 255 may flow from the fluid chamber 272 through the fluid port 328 and into the channel 330. From the channel 330, the heated circulating fluid 255 may flow into the central channel 311 and into the catheter 202. In some examples, the fluid port 328 in the hub manifold may be radially offset (e.g. 180 degrees offset) from the fluid port 287 in the connector manifold 232. The heated treatment fluid 253 may be introduced through the fluid port 252 in the connector assembly and may flow through the channel 288 in the connector manifold 232 directly through the opening 309 into the central channel 311 of the hub manifold 222 or into the circumferentially scaled fluid chamber 274 and then into the central channel 311. The return fluid 251 may be drained or returned from the catheter 202 by flowing through the central channel 311, into the fluid channel 324, and out of the hub manifold 222 through the fluid port 322. In some examples, the treatment fluid, circulating fluid, and return fluid may each be conveyed through separated fluid subchannels either extending through the central channel 311 or through discrete subchannels in the body 312. The subchannels may connect to discrete passages in the catheter 202.

From the fluid port 322, the return fluid 251 may flow into the circumferentially sealed fluid chamber 270. The return fluid 251 may be evacuated from the fluid chamber 270 through the fluid port 285 in the connector manifold 232. From the fluid port 285, the return fluid 251 may flow into the channel 284 and out the fluid port 250 of the connector manifold 232. In some examples, the fluid port 322 in the hub manifold may be radially offset (e.g. 180 degrees offset) from the fluid port 285 in the connector manifold 232. In some examples, the channel 284 for returning the cooled fluid 251 may be spaced away from the channels 286, 288 that convey the heated fluid 255, 253, respectively. The spacing and the insulation provided by the intermediate manifold body 260 or by other insulation features may serve to minimize cooling of the fluid 253, 255 by the cooler fluid 251. Additionally, heat from the fluids 253, 255 may heat the connector and hub manifolds 232, 222 which may help maintain the temperature of the heated fluids and reduce loss from the cooler fluid 251. The heated fluid channels may be separated from the cooled fluid channels in the manifolds to insulate the heated fluid from the cooled, returning fluid. The length of the channels through the manifolds may also be minimized compared to separate tubes to reduce the distance along which heat loss may occur.

Sensors ports 262 may allow access for sensors (not shown) to measure temperature, pressure, chemical composition, or other properties of the heated circulating fluid 255 or the heated treatment fluid 253. The sensors may provide data to a control system (e.g., a control system 812) to adjust parameters of the thermal fluid generation system 122 or the medical instrument system 100.

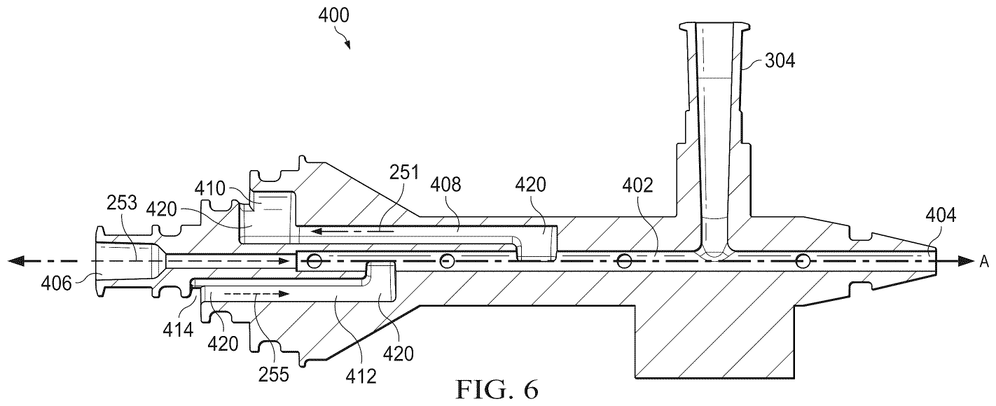

FIG. 6 illustrates an alternative example of a hub manifold 400 that may be used in a hub assembly (e.g. hub assembly 204). The hub manifold 400 may be similar to hub manifold 222, with differences as described. In this example, the hub manifold 400 may include a central fluid channel 402 (e.g., for delivering the heated treatment fluid 253) extending along or parallel to the axis A between a distal opening 404 and a proximal opening 406. A fluid channel 412 (e.g., for delivering the heated circulating fluid 255) may extend between a proximal port 414 and the central fluid channel 402. A fluid channel 408 (e.g., for returning a fluid 521) may extend between a proximal port 410 and the central fluid channel 402. In this example, the fluid channels 412, 408 may be generally parallel to the longitudinal axis A and the central fluid channel 402 with approximately right angle turns 420 to connect to the central fluid channel 402 and the ports 410, 414. In some examples, fluid may be dispensed through the central fluid channel without use of the circulation fluid in the channels 412, 408.

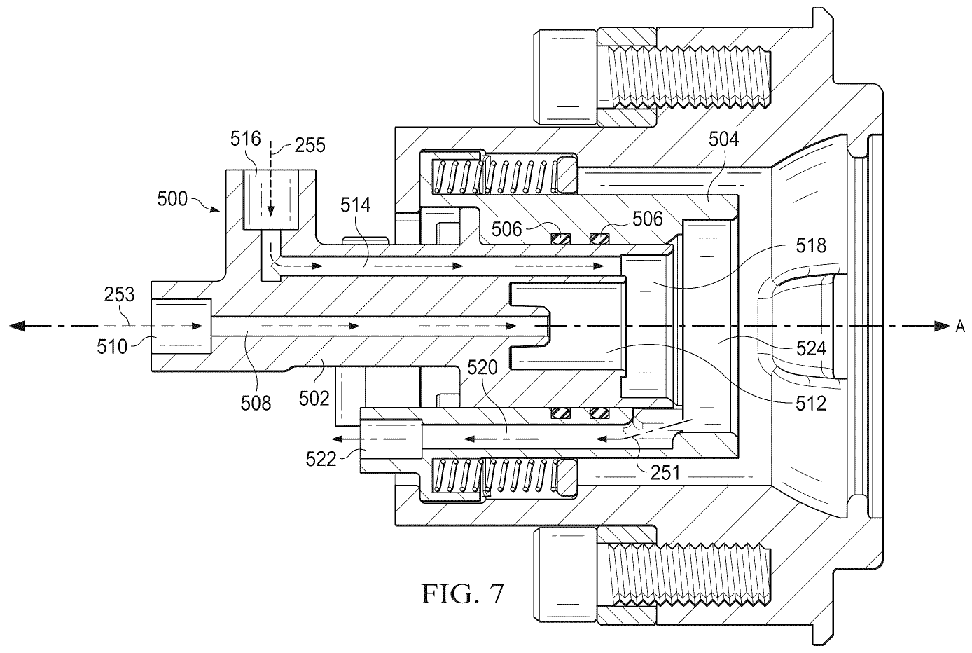

FIG. 7 illustrates an alternative example of a connector manifold 500 that may be used in a connector assembly (e.g., connector assembly 206). The connector manifold 500 may be similar to connector manifold 232, with differences as described. In this example, the connector manifold 500 may include inner manifold body 502 extended within an outer manifold body 504. The inner manifold body 502 and the outer manifold body 504 may be manufactured as separate components. Scaling members 506 such as gaskets or O-rings may provide a fluid-tight seal between the inner and outer manifold bodies 502, 504. In this example, the connector manifold 500 may include a central fluid channel 508 (e.g., for delivering the heated treatment fluid 253) extending along or parallel to the axis A between a proximal opening 510 and a fluid chamber 512. A fluid channel 514 (e.g. for delivering the heated circulating fluid 255) may extend between a proximal port 516 and a fluid chamber 518. A fluid channel 520 (e.g. for returning the fluid 251) may extend between a proximal port 522 and a fluid chamber 524. In this example, the inner manifold body 502 surrounding the fluid channels 508, 514 and the fluid chambers 518, 524 may be formed of a highly conductive material, such as a metal, so that heat is easily transferred between the channel 514 containing the heated circulating fluid 255 and the channel 508 containing the heated treatment fluid 253. In some examples, a heater may be attached to the inner manifold body 502 to help maintain the temperature of the heated circulating and treatment fluids. The outer manifold body 504 may be formed of a highly insulative material, such as a plastic or ceramic, so that the cooled return fluid 251 is isolated from the heated fluids 253, 255 and has minimal or no cooling effect on the heated fluids.

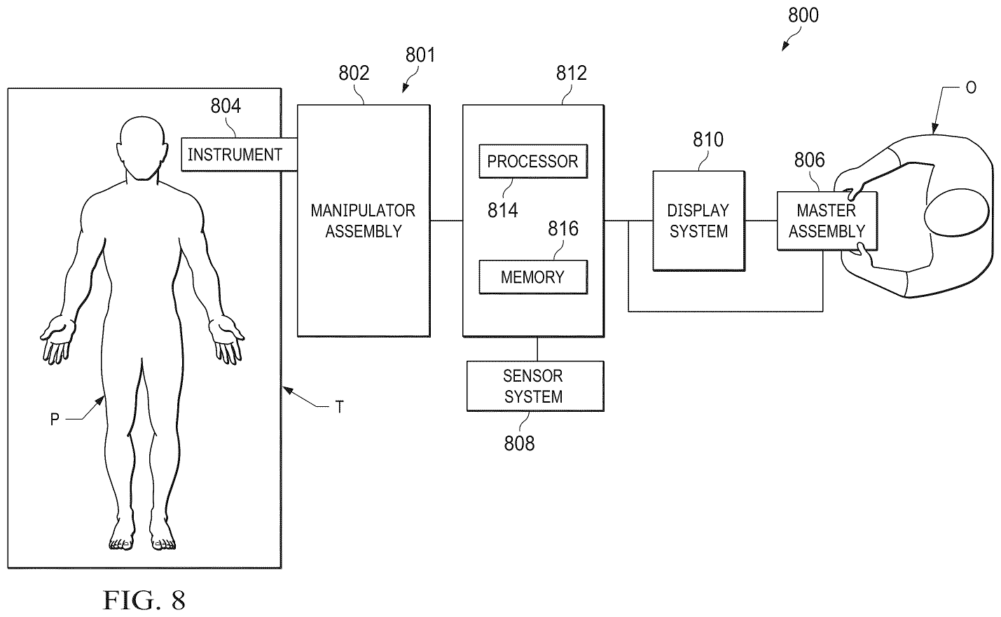

In some examples, the catheter 202 may be delivered through a lumen of an elongated delivery instrument (not shown). The elongated delivery instrument may be an elongate flexible delivery instrument that is steerable via manual or robot-assisted control. For example, the delivery instrument may be a component of a bronchoscope or a navigable, robot-assisted medical instrument system. In other examples, the catheter 202 may be a component of the bronchoscope or a navigable, robotic-assisted medical instrument system. In some examples, the systems and methods disclosed herein may be used in a medical procedure performed with a robot-assisted medical system as described in further detail below. As shown in FIG. 8, a robot-assisted medical system 800 may include a manipulator assembly 802 for operating a medical instrument 804 (e.g., medical instrument system 100, catheter 202, or any of the instruments described herein) in performing various procedures on a patient P positioned on a table T in a surgical environment 801. The manipulator assembly 802 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. A master assembly 806, which may be inside or outside of the surgical environment 801, generally includes one or more control devices for controlling manipulator assembly 802. Manipulator assembly 802 supports medical instrument 804 and may optionally include a plurality of actuators or motors that drive inputs on medical instrument 804 in response to commands from a control system 812. The actuators may optionally include drive systems that when coupled to medical instrument 804 may advance medical instrument 804 into a naturally or surgically created anatomic orifice. Other drive systems may move the distal end of medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). The manipulator assembly 802 may support various other systems for irrigation, treatment, or other purposes. Such systems may include fluid systems (including, for example, reservoirs, heating/cooling elements, pumps, and valves), generators, lasers, interrogators, and ablation components.

Robot-assisted medical system 800 also includes a display system 810 for displaying an image or representation of the surgical site and medical instrument 804 generated by a sensor system 808 which may include an endoscopic imaging system. Display system 810 and master assembly 806 may be oriented so an operator O can control medical instrument 804 and master assembly 806 with the perception of telepresence. Any of the previously described graphical user interfaces may be displayable on a display system 810 and/or a display system of an independent planning workstation.

The sensor system 808 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument 804. The sensor system 808 may also include temperature, pressure, force, or contact sensors or the like.

Robot-assisted medical system 800 may also include control system 812. Control system 812 includes at least one memory 816 and at least one computer processor 814 for effecting control between medical instrument 804, master assembly 806, sensor system 808, and display system 810. Control system 812 also includes programmed instructions (e.g., a non-transitory machine-readable medium storing the instructions) to implement a plurality of operating modes of the robot-assisted medical system including a navigation planning mode, a navigation mode, and/or a procedure mode. Control system 812 also includes programmed instructions (e.g., a non-transitory machine-readable medium storing the instructions) to implement some or all of the processes described in accordance with aspects disclosed herein, including, for example, expanding an expandable device, regulating the temperature of the heating system, regulating valves to control fluid delivery, controlling fluid flow rate, controlling insertion and retraction of the treatment instrument, controlling actuation of a distal end of the treatment instrument, receiving sensor information, altering signals based on the sensor information, selecting a treatment location, and/or determining a size to which the expandable device may be expanded.

Control system 812 may optionally further include a virtual visualization system to provide navigation assistance to operator O when controlling medical instrument 804 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 812 may use a pre-operative image to locate the target tissue (using vision imaging techniques and/or by receiving user input) and create a pre-operative plan, including an optimal first location for performing bronchial passageway and vasculature occlusion. The pre-operative plan may include, for example, a planned size to expand the expandable device, a treatment duration, a treatment temperature, and/or multiple deployment locations.

In the description, specific details have been set forth describing some examples. Numerous specific details are set forth to provide a thorough understanding of the examples. It will be apparent, however, to one skilled in the art that some examples may be practiced without some or all these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions. Not all the illustrated processes may be performed in all examples of the disclosed methods. Additionally, one or more processes that are not expressly illustrated in may be included before, after, in between, or as part of the illustrated processes. In some examples, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in examples of this disclosure may be implemented in software to execute on a processor of a computer system such as control processing system. When implemented in software, the elements of the examples of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative examples of the invention have been described and shown in the accompanying drawings, it is to be understood that such examples are merely illustrative of and not restrictive on the broad invention, and that the examples of the invention are not limited to the specific constructions and arrangements shown and described, since various other modifications may occur to those ordinarily skilled in the art.

What is claimed is:

1. A connector assembly comprising:
a connector housing and
a connector manifold disposed within the connector housing, the connector manifold including,
a manifold body;
a first channel extending through the manifold body to a first chamber;
a second channel extending through the manifold body to a second chamber; and
a third channel extending through the manifold body to a third chamber, wherein the first chamber, the second chamber, and the third chamber are coaxial.

2. The connector assembly of claim 1 further comprising a spring-loaded interlock assembly.

3. The connector assembly of claim 1, wherein the manifold body includes an inner manifold body and an outer manifold body.

4. The connector assembly of claim 3, wherein the inner manifold body is made of a conductive material and the outer manifold body is made of an insulative material.

5. The connector assembly of claim 1, further comprising a fluid temperature sensor.

6. The connector assembly of claim 1, further comprising a first sealing member between the first and second chambers and comprising a second sealing member between the second and third chambers.

7. The connector assembly of claim 6, wherein the first and second sealing members include an o-ring.

8. The connector assembly of claim 1, wherein the first chamber, the second chamber, and the third chamber are sequentially aligned along a longitudinal axis.

9. The connector assembly of claim 1, wherein the first chamber has a larger diameter than the second chamber.

10. The connector assembly of claim 1, further comprising a connector ring, wherein the connector housing includes a first housing portion and a second housing portion, and the connector ring is coupled to the first and second housing portions to connect the first and second housing portions.

11. A catheter assembly comprising:
a catheter body and
a catheter hub assembly coupled to a proximal end of the catheter body, the catheter hub assembly including,
a manifold body including a central fluid channel, a first flange concentric with the central fluid channel, a second flange proximal to the first flange and concentric with the central fluid channel, and a third flange proximal to the second flange and concentric with the central fluid channel;
a first channel extending through the manifold body between the first flange and the central fluid channel; and
a second channel extending through the manifold body between the second flange and the central fluid channel.

12. The catheter assembly of claim 11, wherein the manifold body includes a first circumferential groove distal of the first flange sized to receive a first sealing member, a second circumferential groove distal of the second flange sized to receive a second sealing member, and a third circumferential groove distal of the third flange sized to receive a third sealing member.

13. The catheter assembly of claim 11, wherein the first channel extends at an acute angle to the central fluid channel.

14. The catheter assembly of claim 11, wherein the second channel extends at an acute angle to the central fluid channel.

15. The catheter assembly of claim 11, wherein a portion of at least one of the first and second channels extends parallel to the central channel.

16. The catheter assembly of claim 11, wherein the first flange includes a port to the first channel and wherein the port has an elongated arcuate shape.

17. The catheter assembly of claim 11, wherein the second flange includes a port to the second channel and wherein the port has an elongated arcuate shape.

18. The catheter assembly of claim 11, wherein the first flange has a first flange diameter and the second flange has a second flange diameter, wherein the first flange diameter is larger than the second flange diameter.

19. The catheter assembly of claim 18, wherein the third flange has a third flange diameter and the second flange diameter is larger than the third flange diameter.

20. A fluid connection apparatus, comprising:
a connector assembly configured to connect with a thermal fluid generation system, the connector assembly including:
a first fluid channel and
a second fluid channel; and
a hub assembly configured to connect with an ablation tool, the hub assembly comprising:
a third fluid channel connected to the first fluid channel and
a fourth fluid channel connected to the second fluid channel,
wherein the hub assembly and the connector assembly are configured to rotate with respect to each other while maintaining connections for fluid transfer between (a) the first and the third fluid channels and (b) the second and forth fluid channels.

21. The fluid connection apparatus of claim 20, wherein the first fluid channel and the third fluid channel are connected to provide passage for a return fluid from a catheter coupled to the hub assembly.

22. The fluid connection apparatus of claim 20, wherein the second fluid channel and the fourth fluid channel are connected to provide passage for a circulating heated fluid.

23. The fluid connection apparatus of claim 20, wherein the connector assembly is configured to directly couple with the thermal fluid generation system.

24. The fluid connection apparatus of claim 20, wherein the hub assembly is configured to directly couple with an elongated catheter body.

25. The fluid connection apparatus of claim 20, wherein the connector assembly further includes a spring-loaded interlock assembly including a sensor indicating engagement between the connector assembly and the hub assembly.

26. The fluid connection apparatus of claim 20, wherein the connector assembly includes a connector manifold through which the first and second fluid channels extend and wherein the connector manifold includes an inner manifold body and an outer manifold body.

27. The fluid connection apparatus of claim 26, wherein the inner manifold body is made of a conductive material and the outer manifold body is made of an insulative material.

28. The fluid connection apparatus of claim 20,
wherein the connector assembly includes a connector manifold including a first fluid chamber and a second fluid chamber,
wherein the fluid transfer between the first fluid channel and the third fluid channel is via the first fluid chamber, and
wherein the fluid transfer between the second and the fourth fluid channels is via the second fluid chamber.

29. The fluid connection apparatus of claim 28, wherein the first fluid chamber is sealed from the second fluid chamber by a sealing member.

30. The fluid connection apparatus of claim 29, wherein the connector manifold includes a first circumferential groove sized to receive the sealing member.

31. The fluid connection apparatus of claim 28, wherein the first fluid chamber has a larger diameter than the second fluid chamber.

32. The fluid connection apparatus of claim 28, wherein the first fluid chamber and the second fluid chamber are coaxial about a longitudinal axis.

33. The fluid connection apparatus of claim 20, wherein the connector assembly further includes a connector central fluid channel, and the hub assembly further includes a hub central fluid channel and wherein the connector central fluid channel is connected to the hub central fluid channel.

34. The fluid connection apparatus of claim 33,
wherein the connector assembly includes a central fluid chamber and wherein fluid transfer between the connector central fluid channel and the hub central fluid channel is via the central fluid chamber.

35. The fluid connection apparatus of claim 34, wherein the central fluid chamber is generally coaxial with a longitudinal axis about which hub assembly and the connector assembly rotate.

36. The fluid connection apparatus of claim 33, wherein the connector central fluid channel is connected with the hub central fluid channel to provide passage for a heated treatment fluid.

37. The fluid connection apparatus of claim 33, wherein the third and fourth fluid channels extend at acute angles to the hub central fluid channel.

38. The fluid connection apparatus of claim 33, wherein a portion of at least one of the third and fourth fluid channels extends parallel to the hub central fluid channel.

39. The fluid connection apparatus of claim 20, wherein a port in the hub assembly to the third fluid channel has an elongated arcuate shape.

40. The fluid connection apparatus of claim 20, wherein a port in the hub assembly to the fourth fluid channel has an elongated arcuate shape.
